(19) United States
(12) Patent Application Publication
(10) Pub. No.: US 2021/0267934 A1
(43) Pub. Date: Sep. 2, 2021

(54) CANNABINOID ORAL DISPERSIBLE FILM STRIP

(71) Applicant: Rapid Dose Therapeutics Corp., Burlingtong (CA)

(72) Inventors: JAMES BENJAMIN MACPHAIL, Burlington (CA); RINA CARLINI, Burlington (CA); JAMES LEWIS, Burlington (CA); EMILY QUINN KAPTEYN, Burlington (CA)

(21) Appl. No.: 17/260,712

(22) PCT Filed: Jul. 15, 2019
(86) PCT No.: PCT/CA19/50976

§ 371 Date: Jan. 15, 2021

Related U.S. Application Data
(60) Provisional application No. 62/698,182, filed on Jul. 15, 2018.
(60) Provisional application No. 62/848,555, filed on May 15, 2019.
(60) Provisional application No. 62/853,529, filed on May 28, 2019.

(51) Int. Cl. A61K 31/352; A61K 9/70; A61K 9/00; A61K 31/05; A61K 31/192; A61K 31/593; A61K 31/714; A61K 31/12; A61K 31/522; A61K 31/465; A61K 33/26; A61K 31/7008; A61K 31/202; A61K 31/4045; A61K 31/122; A61K 47/26; A61K 47/10; A61K 47/36; A61K 47/20; A61K 47/14; A61K 47/44

(52) U.S. Cl. CPC A61K 31/352; A61K 47/44; A61K 9/006; A61K 31/05; A61K 31/192; A61K 31/593; A61K 31/714; A61K 31/12; A61K 31/522; A61K 31/465; A61K 33/26; A61K 31/7008; A61K 31/202; A61K 31/4045; A61K 31/122; A61K 47/26; A61K 47/10; A61K 47/36; A61K 47/20; A61K 47/14; A61K 9/7007

(57) ABSTRACT

An oral dispersible film composition comprising at least one active ingredient and a film forming agent, as well as a method of manufacture of same. The oral dispersible film composition can be free of gum resin, and can be utilized to administer various active ingredients, including cannabinoids.

### FIELD OF THE INVENTION

The present invention relates to ingestible oral dispersible film strips that are used to deliver active or medicinal compounds, and more particularly to ingestible oral dispersible film strips that are used to deliver cannabinoid compounds alone or in combination with other natural health or therapeutic compounds. The present invention also relates to a process and an apparatus for manufacturing the ingestible oral thin films, methods and properties of their use.

### BACKGROUND OF THE INVENTION

Cannnabis and Cannabinoids

Cannabis products have been consumed in various forms for thousands of years for both therapeutic and recreational purposes. Patients consume cannabis products through various routes of administration that include inhalation of smoke or vapourized cannabinoids, ingestible and oral dissolvable tablets, liquid tinctures of cannabinoid extracst edible food preparations, and ingestible oral films. There are various advantages and disadvantages to each of these routes of administration for cannabinoids. Some of the primary cannabinoids in cannabis are tetrahydrocannabinol, also known as THC, which is known in two forms: i) (−)-Δ⁹-THC, (6aR,10aR)-6,6,9-trimethyl-3-pentyl-6a,7,8,10a-tetrahydro-6H-benzo[c]chromen-1-ol) and ii) Δ⁸-THC, (6,6,9-trimethyl-3-pentyl-6a,7,10,10a-tetrahydrobenzo[c]chromen-1-ol); cannabidiol, also known as CBD (2-((1R,6R)-3-methyl-6-(prop-1-en-2-yl)cyclohex-2-enyl)-5-pentylbenzene-1,3-diol); cannabinol, also known as CBN (6,6,9-trimethyl-3-pentylbenzo[c]chromen-1-ol); cannabigerol, CBG (2-[(2E)-3,7-dimethylocta-2,6-dienyl]-5-pentylbenzene-1,3-diol); tetrahydrocannabivarin, THCV (6aR,10aR)-6,6,9-trimethyl-3-propyl-6a,7,8,10a-tetrahydrobenzo[c]chromen-1-ol); cannabidivarin, CBDV (2-[(1R,6R)-3-methyl-6-prop-1-en-2-ylcyclohex-2-en-1-yl]-5-propylbenzene-1,3-diol); cannabidiorcinol, CBDO (5-methyl-2-[(1˜{R},6˜{R})-3-methyl-6-prop-1-en-2-ylcyclohex-2-en-1-yl]benzene-1,3-diol) and cannabichromene, CBC (2-methyl-2-(4-methylpent-3-enyl)-7-pentylchromen-5-ol).

Certain of these compounds are found at least in part in their carboxylic acid forms, for example, THC as tetrahydrocannabinolic acid (THCA; (6aR,10aR)-2-carboxy-6,6,9-trimethyl-3-pentyl-6a,7,8,10a-tetrahydrobenzo[c]chromen-1-olate), CBD as cannabidiolic acid (CBDA; 2,4-dihydroxy-3-[(1R,6R)-3-methyl-6-prop-1-en-2-ylcyclohex-2-en-1-yl]-6-pentylbenzoic acid), and CBDV as cannabidivarinic acid (CBDVA; 2,4-dihydroxy-3-[(1R,6R)-3-methyl-6-prop-1-en-2-ylcyclohex-2-en-1-yl]-6-propylbenzoic acid).

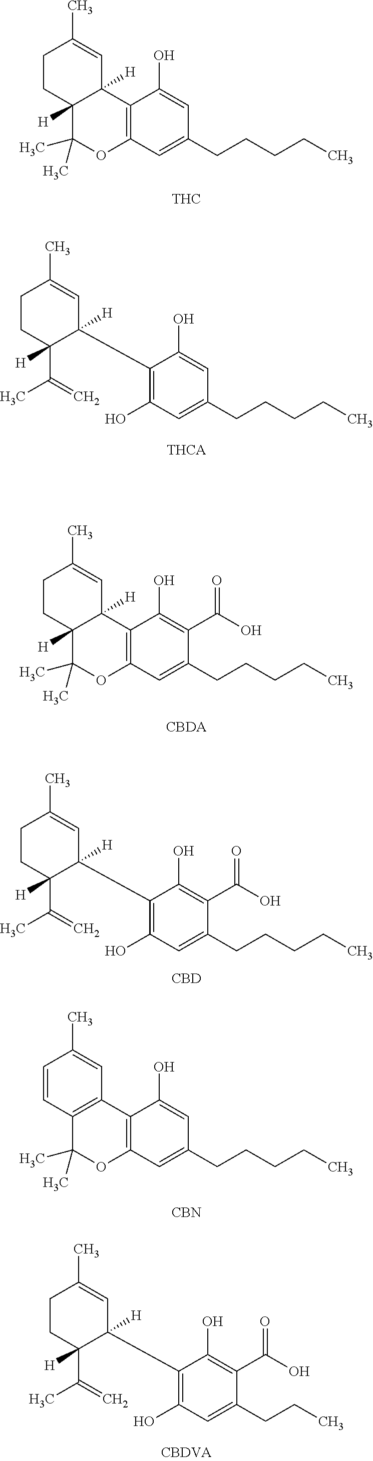

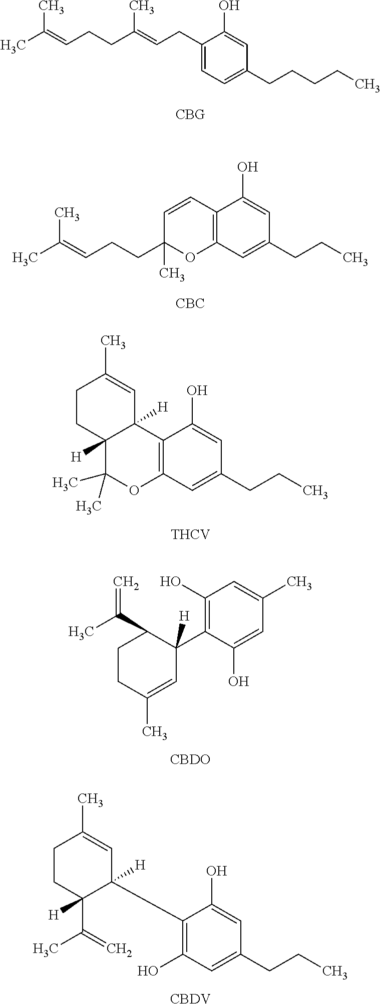

THC, or Delta-9-tetrahydrocannabinol, along with its metabolite, 11-OH-THC, are the principal psychoactive constituents of cannabis, each of which is a partial agonist of the cannabinoid receptors CB1 and CB2, both found in the human endocannabinoid system. Because THC is an illegal drug in many countries, clinical research about the medical use of this compound has been limited and often anecedotal in nature. The American Cancer Society has reported that patients with kidney cancer have required less pain medication when it was combined with cannabis extracts containing THC. Smoking cannabis has been found to alleviate nausea and vomiting in chemotherapy patients. Certain patients with suppressed appetite, including patients taking HIV drugs, have reported that smoking cannabis has improved and promoted food intake. Cannabis has also been documented as a pain relief agent, when inhaled. Cannabidiol (CBD) is one of the prominent active metabolites found in Cannabis plants, both the Cannabis sativa and Cannabis indica species. CBD has been reported to provide various therapeutic benefits such as antioxidant, anti-inflammatory, anti-anxiety and anti-epileptic properties, and is not known to cause psychotropic effects to users of CBD.

In contrast to THC, the delivery of CBD is not known to cause psychotropic effects on its own, though it may attenuate effects of THC. CBD appears to act as an indirect antagonist of cannabinoid agonists, but does not appear to act at the CB1 and CB2 receptors, instead possibly acting as a 5HT1a receptor agonist. The recently published US report in 2017 by the National Academies of Science, Engineering and Medicine entitled Health Benefits of Cannabis and Cannabinoids, summarized the current understanding on the therapeutic use of cannabinoids such as THC and CBD, and highlighted several recommendation for their use in treating a variety of health problems and diseases.

CBD and THC have been purified to high purity, and, like many natural products of therapeutic interest, have also been synthetically and partially-synthetically produced.

Vitamin B12

Vitamin B12 is an essential vitamin, found in many foods. It has been used therapeutically for memory loss, Alzheimer's disease, to slow aging, and to boost cognitive brain function. Vitamin B12 has been found to decrease the risk of ischemia following heart surgery. It has also been used to treat amyotrophic lateral sclerosis (ALS), multiple sclerosis, preventing age-related macular degeneration, treatment of a variety of cancers, chronic obstructive pulmonary disease, and various other diseases and chronic health conditions, including preventing blood clots, shingles, cyanide poisoning, chronic fatigue, liver and kidney disease, and even the treatment of canker sores. It is used topically for psoriasis, and nasally for pernicious anemia. Being an essential vitamin, vitamin B12 deficiency is not uncommon for people who are managing other chronic health conditions such as Inflammatory Bowel Disease, more specifically Crohn's Disease, and other metabolic and cardiovascular diseases.

Methylcobalamin (MeB12) is a cobalamin, a form of vitamin B12. It is believed to be equivalent physiologically to vitamin B12, and is used as a therapeutic substitute for vitamin B12. MeB12 is used in the treatment of peripheral neuropathy, diabetic neuropathy, and as a preliminary treatment for amyotrophic lateral sclerosis.

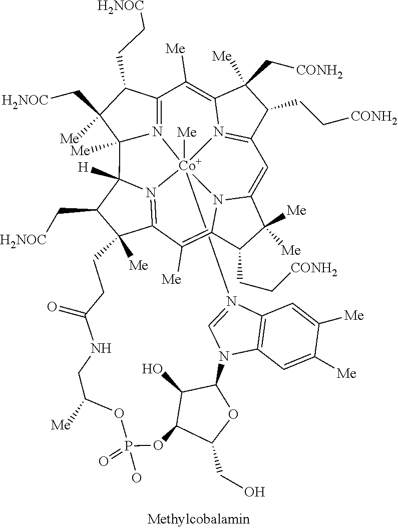

Melatonin

Melatonin is a hormone believed to regulate the circadian cycle in humans, which is the biochemical process that regulates the sleep and awake cycles in humans. It is used as a supplement, for the short-term treatment of jet lag due to travel across large time zone differences, or for regulating sleep and awake cycles for people who have irregular work shifts in the night-time hours.

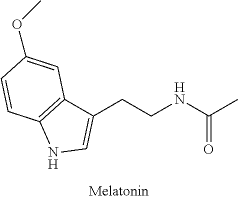 [SMILES: COc1ccc2[nH]cc(CCNC(C)=O)c2c1]

Route of Administration

Route of administration is well known to affect the absorption, distribution, and metabolism parameters of pharmaceutical compounds. Drug delivery via intravenous (i.v.) injection is known to permit relatively rapid onset of therapeutic effects, however, i.v. injection is not always practical outside of a clinical setting. Oral administration is convenient, yet it typically has a very slow onset of therapeutic effects, and drug potency can be lost due to action of the digestive system and/or first pass metabolism.

Oral administration of medicinal and therapeutic agents that occur by absorption across the buccal or sublingual mucosa is an attractive alternative to standard oral administration by ingestion into the gastro-intestinal tract, as it can bypass first pass metabolism in the liver as well as degradation in the digestive tract. The buccal and sublingual mucosa both receive abundant blood supply, and have relatively high permeability allowing drugs to penetrate into the bloodstream and act rapidly. Important practical applications for administration via the oral mucosa include emergency care situations where rapid administration of drugs by non-skilled personnel could be life-saving; in unconscious patients who may have overdosed or experienced a seizure; in elderly dementia patients with dysphagia where swallowing is impaired; as a facile and convenient route of administration of medication to young children; and for drug delivery of medication to animals.

Oral Dispersible/Dissovable Film

Mucosally dissolvable, oral dispersible films are known. See for example US publication 2017/0290870 A1, incorporated herein by reference, which teaches a film strip having cannabinoids as an active ingredient, made by layering an emulsion of the CBD oil onto a structural matrix. See also U.S. Pat. Nos. 10,272,125, 9,717,682, 9,301,948, 8,865,202, 8,735,374, 6,709,671, 6,177,096, and US patent publications US2018/0360736A1, US2018/0078549A1, US2017/0239172A1, US2017/0157119A1, US2016/0220480A1, US2016/0213624A1, US2013/0137698A1, and US2011/0136815A1, all incorporated herein by reference.

It would be desirable to have a mucosally dissolvable, oral dispersible film strip having improved absorption properties. It would be desirable to have a mucosally dissolvable, oral dispersible film strip having improved accuracy and variability of active ingredient dosage.

### SUMMARY OF THE INVENTION

According to one aspect of the present invention is provided an oral dispersible film composition comprising at least one active ingredient and a film forming agent, wherein the active ingredient is dispersed within the film and has an average particle size of 1-20 microns, preferably 5-15 microns, wherein the composition consists of a single layer.

According to certain embodiments, the oral dispersible film is comprised of distribution of particle sizes having polydispersity value of less than 1.5.

According to a further aspect of the present invention is provided an oral dispersible film composition comprising at least one active ingredient and a film forming agent, having a percentage relative standard deviation of potency concentration of the at least one active ingredient in a batch of said oral dispersible film composition of less than 2.5%, preferably less than 1%, more preferably less than 0.5%.

According to one aspect of the present invention is provided an oral dispersible film composition comprising at least one active ingredient and a film forming agent, having a dissolution time in phosphate buffered saline solution at pH 6.8 and 37 degrees C. of less than about 1 minute, preferably less than about 30 seconds, more preferably less than about 20 seconds.

According to certain embodiments, the oral dispersible film composition of any preceding claim wherein the composition contains less than 1 wt %, more preferably less than 0.5%, most preferably 0 wt % of a gum resin. According to certain embodiments, the oral dispersible film composition of any preceding claim wherein the at least one active ingredient is selected from the group of cannabinoid compounds and terpenoid compounds comprised of cannabidiol, delta-9-tetrahydrocannabinol, delta-8-tetrahydrocannabinol, cannabigerol, cannabidiol acid, tetrahydrocannabinol acid, olivetol and olivetol acid or esters.

According to certain embodiments, the at least one active cannabinoid compound is derived biosynthetically from Cannabis plant species, from hemp, from plant-based or animal cell microorganisms, or is obtained by chemical synthesis from non-natural starting compounds.

According to certain embodiments, the oral dispersible film composition comprises a second active ingredient.

According to certain embodiments, the second active ingredient is a nutraceutical or natural health product selected from the group consisting of vitamin D3 (cholecalciferol), docosahexenoic acid (DHA), caffeine, nicotine, ubiquinone (coenzyme Q10), curcumin, natural antioxidants, glucosamine, melatonin, vitamin B12,a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

According to certain embodiments, the film forming agent is selected from the group consisting of pullulan, substituted and modified cellulosic polymers such as HPMC and CMC, alginate salts, starches, pectins, dextrins, gelatins, glycogen, poly(vinylalcohol) and its derivatives including polyvinylacetate, polyethyleneoxide, polyethyleneglycol, polyvinylpyrrolidone (povidone), and the oral film composition preferably contains less than 1 wt %, more preferably less than 0.5 wt %, most preferably 0 wt % of a gum resin.

According to one aspect of the present invention is provided an oral dispersible film composition comprising pullulan, a cannabinoid, glycerol, Tween 80, Span 80, MCT oil, peppermint oil, sucralose, maltodextrin, sorbitol, dimethyl sulfone, and optionally one or more of colourant, flavouring, limonene and menthol. In certain embodiments, the active cannabinoid ingredient is CBD (cannabidiol) or delta-9-THC (delta-9-tetrahydrocannabinol), or blends of CBD and THC, preferably present in a relative weight ratio of between 20:1 to 1:5 (CBD:THC).

According to one aspect of the present invention is provided an oral dispersible film composition, consisting essentially of: a pharmaceutical-grade or food-grade plasticizer (preferably glycerol), a cannabinoid (preferably CBD or delta-9-THC or a blend of CBD and THC in a relative weight ratio of between 20:1 to 1:5), a carrier fluid, a gum-free film forming agent (preferably pullulan), and optionally one or more of a colourant, a flavouring agent, a sweetener, one or more surfactants (preferably non-ionic surfactant), and a permeation enhancer.

According to certain embodiments, the oral dispersible film composition consists essentially of 3-5 wt % plasticizer, 12-18 wt % cannabinoid, 2-8 wt % carrier oil, 40-65 wt % gum-free film forming agent, 4-12 wt % surfactant, 0.1-1 wt % permeation enhancer, and optionally colourant, flavouring, sweetener, limonene and menthol.

According to one aspect of the present invention, the oral dispersible film composition further consists essentially of one or more of vitamin D3 (cholecalciferol), docosahexenoic acid (DHA), caffeine, nicotine, ubiquinone (coenzyme Q10), curcumin, natural antioxidants, glucosamine, melatonin, vitamin B12, a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

According to one aspect of the present invention, the cannabinoid is delta-9-THC.

According to one aspect of the present invention, the cannabinoid is CBD.

According to one aspect of the present invention, the composition consists of a single layer.

According to one aspect of the present invention, the at least one active ingredient is CBD and the second active ingredient is vitamin B12, a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

In certain embodiments, the at least one active ingredient is CBD and the second active ingredient is melatonin.

According to one aspect of the present invention is provided a method of treating active inflammatory bowel disease in a patient in need thereof comprising administering a therapeutically effective amount of an oral dispersible film composition as hereindescribed.

According to one aspect of the present invention is provided a method of treating a sleep disorder or a sleep disturbance such as sleep apnea and insomnia, in a patient in need thereof comprising administering a therapeutically effective amount of an oral dispersible film composition as hereindescribed.

According to one aspect of the present invention is provided a use of an oral dispersible film composition for the treatment of dysfunctional anxiety and pain in patients suffering from post-traumatic stress disorder and similar mental health conditions.

According to one aspect of the present invention is provided a method of manufacturing an oral dispersible film comprising a first active medicinal ingredient, said method comprising: combining the first active ingredient with a carrier fluid, and blending to form a mixture; adding at least one surfactant to the mixture; agitating the mixture at a high shear rate to create a stable dispersion; adding a polymer film-forming agent, a plasticizer, a permeation enhancer, a diluent, optionally at least one flavouring agent, and optionally at least one colorant; casting the stable dispersion on a flexible substrate having thickness in the range of 0.02 mm to 0.08 mm; drying the dispersion on the flexible substrate at a local temperature ranging from 70° C. to no more than 100° C. and relative humidity ranging from 40% to about 55%, to form the oral dispersible thin film.

In certain embodiments, the agitating at a high shear rate results in the mixture having an average particle size of the one or more active ingredient being in the range of 1 to 20 microns, and a polydispersity value of less than 1.5.

In certain embodiments, the first active ingredient is a cannabinoid.

In certain embodiments, the first active ingredient is CBD.

In certain embodiments, the first active ingredient is THCA.

In certain embodiments, the first active ingredient is THC.

According to one aspect of the present invention is provided the method as herein described, wherein the oral dispersible film comprises a second active ingredient, said method comprising combining the second active pharmaceutical ingredient with the carrier before, simultaneously with, or after, the combining of the first active pharmaceutical ingredient with the carrier.

In certain embodiments, the second active ingredient is selected from the group consisting of vitamin D3 (cholecalciferol), docosahexenoic acid (DHA), caffeine, nicotine, ubiquinone (coenzyme Q10), curcumin, natural antioxidants, glucosamine, melatonin, vitamin B12, a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

In certain embodiments, the second active ingredient is melatonin.

In certain embodiments, the second active ingredient is vitamin B12 or methylcobalamin.

In certain embodiments, the first active ingredient is CBD and the second active ingredient is melatonin.

In certain embodiments, the first active ingredient is CBD and the second active ingredient is vitamin B12, a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

According to one aspect of the present invention is provided a method as herein described, wherein the polymer film-forming agent comprises less than 1 wt %, more preferably less than 0.5 wt % of gum resin, and even more preferably is free of gum resin.

According to one aspect of the present invention is provided an oral dispersible film manufactured utilizing the method as herein described.

According to one aspect of the present invention is provided an oral dispersible film manufactured utilizing the method as herein described, wherein the particle dispersion within the film has an average particle size of 1-20 microns, preferably 5-15 microns.

According to one aspect of the present invention is provided an oral dispersible film composition manufactured utilizing the method of any one of the preceding claims, having a percentage relative standard deviation of potency concentration of the at least one active ingredient in a batch of said oral dispersible film composition of less than 2.5%, preferably less than 1%, more preferably less than 0.5%.

According to one aspect of the present invention is provided an oral dispersible film composition manufactured utilizing the method of any one of the preceding claims, having a dissolution time in phosphate buffered saline solution at pH 6.8 and 37 degrees C. of less than about 1 minute, preferably less than about 30 seconds, more preferably less than about 20 seconds.

According to a further aspect of the present invention is provided an oral dispersible film composition manufactured utilizing the method as herein described, wherein the distribution of particle sizes having polydispersity value of less than 1.5.

According to a further aspect of the present invention is provided the use of an oral dispersible film composition manufactured utilizing the method as herein described in the treatment of active inflammatory bowel disease, wherein the active ingredient is CBD and the optional second active ingredient is vitamin B12, a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

According to a further aspect of the present invention is provided the use of an oral dispersible film composition manufactured utilizing the method of any one of the preceding claims in the treatment of a sleep disorder or a sleep disturbance, wherein the active ingredient is CBD and the optional second active ingredient is melatonin.

According to a further aspect of the present invention is provided the use of an oral dispersible film composition as herein described in the treatment of active inflammatory bowel disease, wherein the active ingredient is CBD and the second active ingredient is vitamin B12, a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

According to a further aspect of the present invention is provided the use of an oral dispersible film composition as herein described in the treatment of a sleep disorder or a sleep disturbance, wherein the active ingredient is CBD and the second active ingredient is melatonin.

According to a further aspect of the present invention is provided an oral dispersible film composition of any one of the preceding claims, or an oral dispersible film composition manufactured utilizing the method of any one of the preceding claims, having improved bioavailability by a factor of 1.5 or more compared to oral administration directly into the GI tract.

### BRIEF DESCRIPTION OF THE DRAWINGS

The invention will be described in further detail having regard to the drawings in which:

FIG. 1 is a block diagram showing a method according to an embodiment of the invention;

FIG. 2 is a schematic representation of a part of a method according to an embodiment of the invention;

FIGS. 3A-3B are schematic representations of a part of a method according to an embodiment of the invention; and

FIG. 4 is a schematic representation of a part of a method according to an embodiment of the invention.

### DETAILED DESCRIPTION OF THE INVENTION

A better understanding of the present invention and its objects and advantages will become apparent to those skilled in this art from the following detailed description, wherein there is described only the preferred embodiment of the invention, simply by way of illustration of the best mode contemplated for carrying out the invention. As will be realized, the invention is capable of modifications in various obvious respects, all without departing from the scope and spirit of the invention. Accordingly, the description should be regarded as illustrative in nature and not as restrictive.

As used herein, the terms “oral dispersible film strip”, “strip”, “film strip” and “film” refer to sheets of variable dimensions comprising a polymeric carrier matrix, and having any shape, including rectangular, square, or other desired shape. The films described herein are typically thin films with thickness that can range from about 30 microns to about 300 microns, for example from about 50 micron to about 200 microns, such as from 60 to 100 microns, but may be any desired thickness and size so long as they can be placed comfortably into the oral cavity of the user. Films are for example a single layer and are typically inherently flexible to enable rapid dissolution in the mouth, permeation through the oral mucosa and entry into the bloodstream via capillaries; ideally not stiff wafers that must be masticated into smaller portions since these have a higher probability of being swallowed and subsequently digested in the GI tract.

According to an embodiment of the invention, the oral dispersible film strip can contain at least one active ingredient. Preferably, the active ingredient is a cannabinoid. The term “cannabinoid” refers to a naturally extracted, biosynthetic or chemically synthetic compounds having the same chemical structure as the key metabolites of the Cannabis sativa or Cannabis indica plants, and that have physiological activity as agonists of one or more cannabinoid receptors, such as the CB₁ and CB₂ receptors. The preferred cannabinoids for use in the oral dispersible film strips can be seleted from one or more of the following compounds: tetrahydrocannabinol (THC), tetrahydrocannabinolic acid (THCA), cannabidiol (CBD), cannabigerol (CBG) and cannabinol (CBN). Any form of cannabinoid may be used, for example, a purified crystalline powder, a non-crystalline powder, an oil suspension or oil solution, or even a crude plant extract. In preferable embodiments, a high purity powder (for example, greater than 90%, such as 95% or even 99%) of CBD is used. It has been found that high purity powders, such as synthetically manufactured, pure cannabinoid powders having greater than 99% purity of a single cannabinoid, are particularly advantageous for the therapeutic applications claimed herein.

In another embodiment, the oral dispersible film strip can include more than one active ingredient. Such an oral dispersible film strip will allow for co-administration of active ingredients to provide one or more health or therapeutic benefits, which can be unrelated, complimentary, additive or synergistic. The second active ingredient may include any pharmaceutical, nutraceutical, vitamin, hormone, or therapeutic compound. Interestingly, even though cannabinoids are notoriously hydrophobic, the second active ingredient may be a hydrophobic, hydrophilic, or water soluble ingredient. In certain embodiments, the further active ingredient is vitamin D₃ (Cholecalciferol), docosahexaenoic acid (DHA), caffeine, nicotine, ubiquinone (coenzyme Q₁₀), glucosamine, melatonin, vitamin B12, or biologically active metabolites, such as methylcobalamin, or any of their derivative forms such as inorganic salts, organic salts or polymer-bound complexes, protein-bound complexes, and similar derivatives.

A surfactant may be used in the oral dispersible film strip. The surfactant may include ionic surfactants including anionic and/or cationic surfactants such as sodium dodecyl sulphate (SDS), sodium lauryl sulfate (SLS), benzalkonium chloride, benzthonium chloride, benzyldimethyldodecylammonium bromide (BDDAB), and non-ionic surfactants such as polysorbate 80, sorbitan monooleate, lecithins, glycolipids, fatty alcohols, fatty acids, esters of fatty alcohols and fatty acids, sorbitan esters, polyols such as polysorbates, sorbitans, long-chain aliphatic acids that can be saturated or unsaturated and having more than 6 carbons, such as stearic acid, lauric acid, oleic acid, lineoleic acid, PEG-40 hydrogenated castor oil, sodium deoxycholate, poloxamers, bile salts such as sodium taurocholate, derivatives thereof, and combinations thereof. Other suitable surfactants could also be used, and preferably, low molecular weight surfactants are advantageous. The amounts used of such surfactants can range from about 0.5 wt % to about 20 wt %, and more preferably between 1 wt % to about 10 wt % of total, for example, 4 wt % to 8 wt % of total, although it is possible to use surfactants that are out of this range.

A plasticizer may be used in the oral dispersible film strip. The plasticizer should be a food-grade or pharmaceutical-grade compound, for example one or more of the include low molecular weight polyols such as glycerol, propylene glycol, polyethylene glycols, monosaccharides such as xylitol, erythritol, mannitol, sorbitol; disaccharides such as sucrose, lactose, and maltose; oligosaccharides such as glycogen, starch, inulin, and dextrins such as maltodextrin and similar compounds; citrate derivatives such as tributyl, triethyl, acetyl citrate, citrate ester, triacetin, castor oil, medium-chain triglycerides (MCT) of fatty acids (also known as MCT oil), and various plant oils of low viscosity such as soybean oil, canola oil, corn oil and similar oils mineral oil, myglyol, derivatives thereof, and combinations thereof.

A carrier fluid or diluent may be used to facilitate solubilization of the active ingredient and/or the film-forming agent. This may be a solubilization agent. The carrier may include medium chain triglyceride (MCT) oil, peppermint oil, hemp oil, coconut oil, or any other edible oil that is Generally Recognized as Safe (GRAS). For example, olive oil, or hemp oil may be used. Other edible oils may be suitable, including major food oils such as vegetable, canola, peanut, almond, coconut, avocado, sesame, corn, cottonseed, palm, safflower, rapeseed, soybean, and sunflower; nut oils such as almond, beech nut, brazil nut, cashew, hazelnut, macadamia, mongongo nut, pecan, pine nut, pistachio, and walnut, citrus oils such as grapefruit seed oil, lemon oil, and orange oil; oils from melon and gourd seeds including from the members of the Cucurbitaceae family, such as bitter gourd oil, bottle gourd oil, buffalo gourd oil, butternut squash seed oil, egusi seed oil (from Cucumeropsis mannii naudin seeds), pumpkin seed oil and watermelon seed oil; food supplement oils such as Agai oil (from fruit of several species of the Agai palm), black seed oil (pressed from Nigella sativa seeds), blackcurrant seed oil (from Ribes nigrum seeds), borage seed oil (from Borago officinalis seeds), evening primrose oil (from Oenothera biennis seeds), and flaxseed oil (or linseed oil) (from Linum usitatissimum seed); or other known edible oils such as amaranth seed oil (from sees of grain amaranth species including Amaranthus cruentus and Amaranthus hypochondriacus), apricot oil, apple seed oil, argan oil (from the seeds of the Argania spinosa), artichoke, avocado, babassu oil (from the seeds of the Attalea speciosa), ben oil (from the seeds of Moringa oleifera), Borneo tallow nut oil (extracted from the fruit of species of genus Shorea), cape chestnut oil (also called yangu oil), carob pod oil (Algaroba oil), cocoa butter, cocklebur oil (from species of genus Xanthium), cohune oil (from Attalea cohune), coriander seed, date seed oil, dika oil (from Irvingia gabo mensis seeds), false flax oil (from Camelina sativa seeds), grape seed oil, hemp oil, kapok seed oil (from Cieba pentandra seeds), kenafseed oil (from Hibiscus cannabinus seeds), lallemantia oil (from Lallemantia iberica seeds), mafura oil (from Trichilia emetica seeds), marula oil (from Sclerocarya birrea kernel), meadowfoam seed oil, mustard oil, niger seed oil (including from Guizotia abyssinica), nutmeg butter (extracted by expression form the fruit of cogeners of genus Myristica), nutmeg oil, okra seed oil (from Abelmoschus esculentus seeds), papaya seed oil, papaya oil produced by maceration, perilla seed oil, persim mon seed oil (including from Diospyros virginiana), pequi oil (from Caryocar basiliense seeds), pili nut oil (from Canarium ovatum seeds), pomegranate seed oil (from Punica granatum seeds), poppy seed oil, pracaxi oil (from Pentaclethra macroloba seeds), prune kernel oil, peach kernel oil, quinoa oil, ramtil oil (from one of several species of genus Guizotia abyssinica seeds), rice bran oil, royle oil (from Prinsepia utilis seeds), Sacha inchi oil, Sapote oil, see oil (from Jessenia bataua seeds), shea butter, taramira oil (from arugula or Eruca sativa seeds), tea seed oil (camelia oil), thistle oil (from Silybum marianum seeds), Tigernut oil (or nut-sedge oil, from the Cyperus esculentus tuber), tobacco seed oil (from Nicotiana species seeds, if purified), tomato seed oil, and wheat germ oil. In certain embodiments, the carrier fluid is (MCT) oil and/or peppermint oil.

In certain embodiments, about 0.1 to 40% w/w of oil is utilized, for example 2.5% to 20% w/w of oil. In certain embodiments between 10:1 to 1:10 weight ratio of cannabinoid:oil is used, for example, about a 3:1 weight ratio.

A film-forming agent, such as a film-forming polymer may be used to form the oral dispersible film strip. The film-forming polymer preferably includes hydroxypropyl methylcellulose (HPMC) or Pullulan. Alternatively, the film may be formed of other synthetic and natural polymers, or include thickening agents, such as acetylated distarch adipate; agar; alginic acid; arrowroot; beta-glucan; calcium alginate; carrageenan; cassia Gum; chondrin; collagen; corn starch; dextrin; disodium phosphate; disodium pyrophosphate; file powder; galactomannan; gelatin; gellan gum; glucomannan; guar gum; gulaman; gum karaya; hydroxypropyl distarch phosphate; hypromellose; Irvingia gabonensis; konjac; kudzu; bean gum; maltodextrin; methyl cellulose; millet jelly; modified starch; Monodora pyristica; monosodium phosphate; mung bean; natural gum; pectin; phosphate distarch; phosphate; polydextrose; potassium 30 bitartrate; potato starch; Psyllium seed husks; sago; salep; flour; sodium phosphate; starch; tapioca; tetrasodium pyrophosphate; tragacanth; waxy corn; methylcellulose; hydroxypropylmethylcellulose; ethylcellulose; sodium alginate; poly (methacrylic acid-co-ethyl acrylate), poly (methacrylic acid co-methyl methacrylate); 30 polyvinylpyrrolidone; polylactic acid (PLA); poly-L-lactide (PLLA); poly-D-lactide (PLDA); poly (lactic co-glycolic acid (PLGA); chitosan; chitin; xanthan gum; glycogen; poly-HEMA; pOEGMA; microcrystalline cellulose; derivatives thereof; and combinations thereof. In certain embodiments, the film-forming agent is used in a range of about 30-80 wt %, for example 45-60 wt %, such as about 50 wt %.

Though the above-listed film forming agents can be used, it has been found that a gum-free formulation is particularly advantageous for the present invention, as it may provide faster dissolution and permeation in the sublingual or buccal oral mucosa, and therefore more rapid delivery of the active ingredients into the bloodstream. For example, a formulation where HPMC and/or pullulan is used as film-forming polymer, but which is free of carrageenan, cassia gum, gellan gum, guar gum, gum karaya, bean gum, xanthan gum, rosin gum, or other natural gums may provide improved performance. The term “gum-free”, when used in this specification, refers to a composition that is substantially or essentially free of such gums.

The term “consisting essentially of”, where used in this specification, means that the composition contains no deliberately added other components, and any unavoidable or incidental additional components are only present, if at all, as impurities in other ingredients and/or in amounts that do not affect relevant measurable properties.

Colouring agents may also be present in the oral dispersible film and could include titanium dioxide, and dyes suitable for food such as those known as F.D. & C. dyes and natural coloring agents such as grape skin extract, beet red powder, beta-carotene, annato, carmine, turmeric, paprika, etc. The colouring agent, if present in the film, may range from about 0 to about 2.5 weight percent of the total composition, for example, 1% w/w of the total composition.

Flavouring agents may also be present in the oral dispersible film and could include synthetic flavour oils and flavouring aromatics and/or natural oils, extracts from plants, leaves, flowers, fruits and so forth and combinations thereof. These may include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Also useful as flavors are vanilla, citrus oil, including lemon, orange, grape, lime and grapefruit, and fruit essences, including apple, pear, peach, strawberry, raspberry, cherry, plum, pineapple, apricot and so forth. Flavours which have been found to be particularly useful include commercially available orange, grape, cherry and bubble gum flavors and mixtures thereof. Flavour acids, such as citric acid, malic acid, tartaric acid, lactic acid, and ascorbic acid, may also be used to provide acidic tastes. Flavours may be present in an amount ranging from about 0.5 to about 6.0 by weight based upon the weight of the composition.

Sweetening agents may also be present in the oral dispersible film and could include natural sweeteners, for example, sucrose, Stevia, corn syrup, maple syrup, erythritol, maltitol, mannitol, dextrose, fructose, xylitol, sorbitol, isomalt, and the like, or combinations thereof; and artificial sweeteners, for example, aspartame, sucralose, acesulfame potassium, saccharin, saccharin cyclamate, and the like, or combinations thereof. The amount of the one or more sweetener agents may be, for example, at least about 0.05 weight percent (wt. %) based on the total weight of the aqueous composition.

The oral dispersible film strip may contain a permeation enhancer. The permeation enhancer may be present at from about 0.001% to about 10% by weight of the film, preferably less than 3% by weight of the film, and may include dimethyl sulfone, calcium chelators, polycarboxylic acids, zonula occluding toxin, poly-L-arginine, chitosan derivatives, niacin, omega 3 or 6 fatty acids or other fatty acids, menthol, sodium caprate, sodium deoxycholate, dipotassium glycyrrhizinate, 25 furanocoumarins and grapefruit derivatives, bile salts, ethylenediaminetetraacetic acid (EDTA), tocopheryl polyethyleneglycol succinate (TPGS), derivatives thereof, and combinations thereof, or the like.

The oral dispersible film strip may also include one or more of a permeability enhancer, a taste masking agent, a bitter blocker, a filler, an effervescent agent, an anti-oxidant, a disintegrating agent, a pH modifying agent, a buffer, a complexing agent, a bioadhesive, a sheet adhesive, an emulsifying agent, a crystallization inhibitor, a preservative, a unique identifying agent such as a UV active fluorophore, and an antimicrobial.

It is contemplated that the dispersible film strips can be generated for animal health use. When creating film strips intended for animal consumption, different flavouring agents may be used to improve the desirability and palatability of the oral dispersible film. Preferred flavouring agents are those that are not derived from animal sources. In various embodiments, flavouring components derived from fruit, meat (including, but not limited to pork, beef, chicken, fish, poultry, and the like), vegetable, cheese, bacon, cheese-bacon and/or artificial flavourings may be used. A flavouring component is typically chosen based upon consideration related to the animal that will be ingesting the thin strip. For example, a horse may prefer an apple flavouring component, while a dog may prefer a meat flavoring component. Although flavouring components derived from non-animal sources are preferred, in some embodiments, natural flavours containing beef or liver extracts, etc., may be used such as braised beef flavour artificial powdered beef flavour, roast beef flavour and corned beef flavour among others. Non-animal flavouring agents include, but are not limited to, artificial beef flavours, flavours derived from plant proteins such as soy protein to which artificial flavouring has been added (e.g. soy-derived bacon flavoring), and flavours derived from plant proteins such as soy protein with no artificial flavouring.

When the dispersible film strips are generated for animal health use, the active ingredient can include any known veterinary pharmaceutical or therapeutic agent or combination. The veterinary dispersible thin strips may or may not include cannabinoids.

The oral dispersible film strips can be used to treat and/or prevent a disease, a disorder, or a condition. The active ingredient in the oral dispersible film strip will dictate the disease, disorder, or condition. Oral dispersible film strips that contain more than one active ingredient may provide synergistic activity when treating and/or preventing the disease, disorder, or condition. Oral dispersible film strips comprising a cannabinoid may be used to treat and/or prevent appetite suppression, neuropathic pain, nausea, intraocular pressure, anxiety, sleep disorders such as insomnia, seizures, and any number of other diseases, disorders, or conditions. Specifically, an oral dispersible film strip comprising a cannabinoid (for example, cannabidiol) and melatonin can be used to treat and/or prevent sleep disorders and sleep disturbances such as sleep apnea and insomnia. An oral dispersible film strip comprising a cannabinoid (for example, CBD) and a B12 vitamin can be useful for treating and/or preventing B12 deficiency and its associated pain, for example.

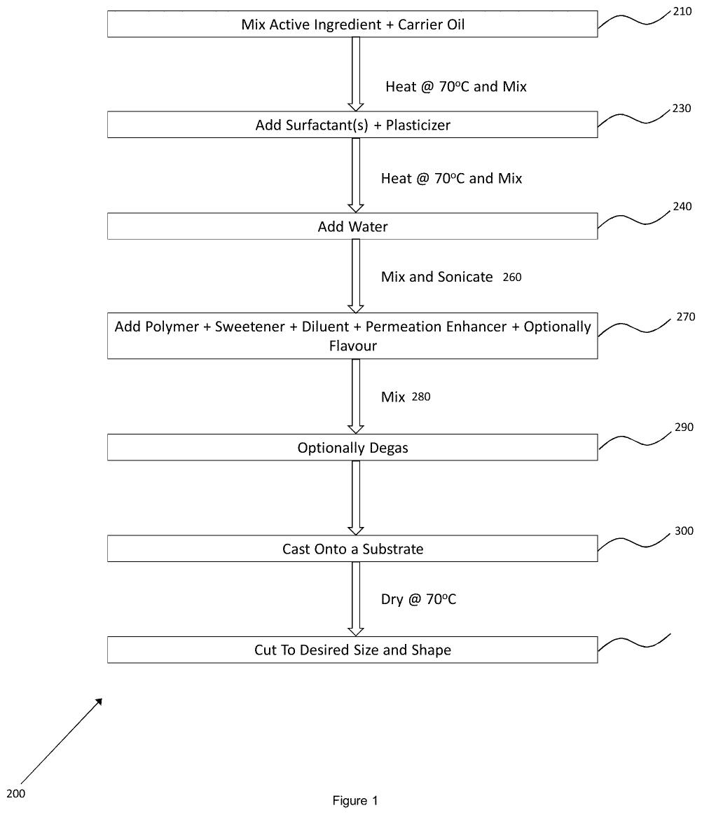

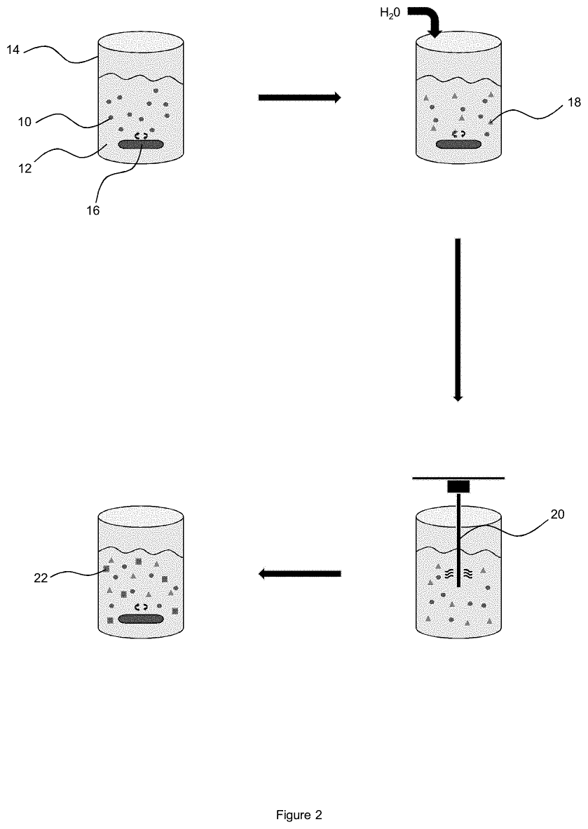

Referring to the Figures, FIG. 1 is an example of a block flow diagram illustrating a method 200 of preparing an oral dispersible film strip according to an embodiment of the invention, and FIGS. 2 to 4 illustrate a schematic diagram of the method. An active ingredient 10, for example, an oleophilic, active ingredient such as a cannabinoid, such as a purified CBD, was added 210 to a carrier 12 in a container 14 forming a first mixture. The mixture was stirred 220, such as with a stir bar 16, at an elevated temperature of 50-90° C., for example about 70° C. a sufficient amount of time for the active ingredient 10 to substantially dissolve or become suspended into the carrier 12, and produce a homogenous mixture. Care was used to keep the elevated temperature below a temperature at which the active ingredient 10 is known to substantially degrade, decompose, or oxidize.

To this homogenous mixture was added 230 a surfactant 18 and optionally a plasticizer. This mixture was stirred 240, such as with a stir bar 16, at an elevated temperature of 50-90° C., for example, about 70° C. a sufficient amount of time for the surfactant 18 and optionally the plasticizer to become dispersed in solution. The mixture underwent brief stirring and was then subjected to further agitation 260 applying high shear, such as by sonication with a probe sonicator 20, a dispersion paddle stirring at high agitation rate of 7000-1000 rpm, to create a stable dispersion.

After agitation 260, a polymer 22, for example, pullulan, was added 270, along with optionally one or a combination of a flavouring agent, a sweetener, a permeation enhancer, and a diluent. The stable dispersion was further mixed 280, such as with a stir bar 16 or a paddle mixer. Optionally, the stable dispersion may be degassed 290, such as by stirring under reduced pressure for example at 15 inHg.

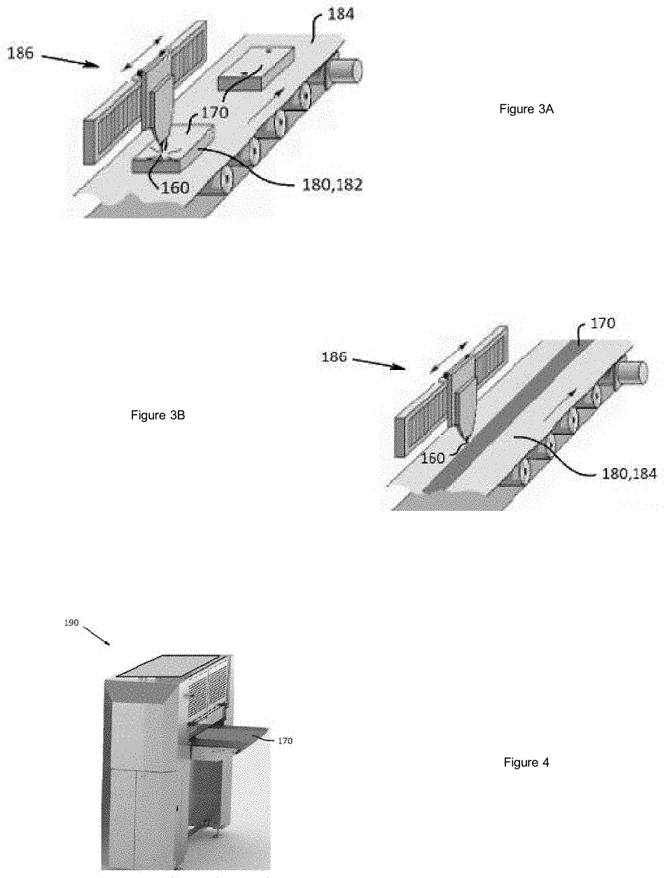

The stable dispersion was then preferably cast 300 to form an oral dispersible film. The oral dispersible film 170 was formed by placing the stable dispersion 160 onto a clean receiving substrate 180 or the like to form the oral dispersible film 170, as can be seen in FIGS. 3A and 3B. The stable dispersion 160 may be dispensed from a suitable apparatus equipped with a doctor blade as would be known in the art, such as a dispensing apparatus 186, onto the receiving substrate 180. The stable dispersion 160 may be poured, injected, or otherwise deposited onto the receiving surface 180 by any suitable process or means. The receiving surface 180 can be of any suitable type, such as a tray 182 on a conveyor belt 184, as shown in FIG. 3A, or a conveyor belt itself 184, as shown in FIG. 3B.

Preferably, the cast stable dispersion 160 was dried 310 by being subjected to heat in order to form the oral dispersible film 170. The apparatus can move the receiving surface 180 through e.g., a convention style oven, or alternatively, the drying can be done in a batch process. In certain embodiments, the cast stable dispersion was air dried at ambient temperature. In other embodiments, the drying was done with a substrate temperature set to 55-80 degrees C., for example about 70 degrees C., with an air flow surrounding the substrate typically hotter than the substrate temperature, for example 100 degrees C. In a typical embodiment, drying was performed for between about 1 and 5 minutes. After the oral dispersible film 170 has been dried, it would have a uniform thickness in the range of 75 to 100 microns. The film can be cut into any desirable shape and size. Alternatively, the stable dispersion may be cast into forms of the desired size and shape, which eliminated the cutting step.

Casting the active ingredient as a stable dispersion as opposed to an emulsion allowed for more even distribution of the active ingredient on the oral dispersible film strip. In one embodiment, the oral dispersible film made with the method of the present invention and having CBD as the active ingredient,w as found to have percent relative standard deviation of the CBD concentration per strip of less than 2.5%, preferably less than 1.0% and more preferably less than 0.5%. This is as compared to prior art methods, where, in the absence of the creation of a stable dispersion during the process, distribution of the active ingredient can be much less uniform in the final product.

In certain embodiments, it is advantageous to have a single layer film strip, as described above. A single layer strip typically has a quicker absorption profile. However, in certain other embodiments, a multi-layer film strip is advantageous. A multi-layer film strip may be advantageous if it is desired to keep one pharmaceutically active ingredient away from a second pharmaceutically active ingredient, for example, where the ingredients may interact or degrade. In such an example, a three layer laminate may be utilized, with an inert layer sandwiched between the two active layers. In other embodiments, it may be advantageous to manufacture a multi-layer film strip having a single active pharmaceutical layer, manufactured as described above, overtop of a muco-adhesive layer, which aids in the adhering of the strip to the oral lingual, sublingual or buccal mucosa of the patient. This may be particularly useful in veterinary applications, as described further, below. A multi-layer film strip may also comprise an abrasive layer for enhancing mucosal, sublingual, or buccal delivery, or permeation enhancers, particularly for animal health applications.

Specific ranges, values, and embodiments provided below are for illustration purposes only and do not otherwise limit the scope of the invention, as defined by the claims. The specific ranges, values, and embodiments described below encompass all combinations and sub-combinations of each disclosed range, value, and embodiment, whether or not expressly described as such. The invention will now be explained more specifically with reference to the following examples, which are given for illustration of the invention and are not intended to be limiting thereof.

### EXAMPLES

### Comparative Example 1

### Prior Art Oral Thin Film Composition and Method

An oral thin film composition and method of making the film was prepared according to U.S. Pat. No. 10,265,362.

A 1000-mL beaker was charged with polysorbate 80 (10.78 g), glycerin (6.34 g), and milli-Q water (104.9 g), forming a clear and colourless “aqueous solution”. CBD isolate (15.05 g) was added to the solution and the resultant opaque, white mixture was heated and stirred until the CBD isolate dissolved (34 min at 63° C.). To the resultant “emulsion” was added pullulan (28.61 g), sucralose (2.00 g), guar gum (4.07 g), pectin (5.08 g), vanilla flavour (13.47 g), peppermint oil (4.47 g), menthol (8.96 g), and green food colouring (2.04 g). The ingredients were combined with stirring until the “modified emulsion” had a smooth and tacky consistency. The modified emulsion was casted into a thin film using a convection style heating chamber equipped with a doctor blade casting assembly at 0.55 m/min, followed by a heating cycle set at 100° C. at 45% RH. The green film was allowed to dry in ambient conditions for an additional 30 min to produce a workable, tacky film.

### Comparative Example 2

### Prior Art Oral Thin Film Method with New Formulation

An oral thin film composition made with the formula of this invention was prepared using the method according to U.S. Pat. No. 10,265,362, for the purpose of comparing two different methods used to make the same oral thin film composition.

A 100-mL beaker was charged with Tween 80 (0.49 g), Span 80 (0.45 g), glycerol (0.41 g), and deionized water (15.47 g), forming a clear and colourless “solution”. CBD isolate (1.7 g, 95% purity) was added to the solution and the resulting white mixture was heated and stirred for 1 hour at 65° C. until the CBD isolate was dissolved. To this mixture (the “emulsion”) was added pullulan (6.36 g), sucralose (0.01 g), maltodextrin (0.67 g), sorbitol (0.68 g), dimethyl sulfone (0.06 g), MCT oil (0.3 g), and peppermint oil (0.29 g). The ingredients were combined and stirred to produce a “modified emulsion” which had uniform consistency. Using a mechanical casting apparatus equipped with doctor blade assembly and convection heating chamber, the modified emulsion was cast into a thin film onto a PET substrate at the rate of 0.55 m/min, followed by a heating cycle set at 100° C. at 45% RH, which produced an off-white thin film that was subsequently cut into strips.

General Procedure for Making Cannabinoid Oral Dispersible Thin Film

A 600 mL beaker equipped with a magnetic stir bar is added CBD (powder form), MCT oil, and peppermint oil. The mixture is heated at a minimum of 70° C. while stirring until the mixture becomes a homogenous, transparent amber-colored liquid. To the beaker are added glycerol, Tween 80, Span 80 and the mixture stirred at a minimum of 70° C. until dissolved. Sufficient deionized H₂O is added to the mixture so as to achieve 40-45 wt % w/w solids content, and the resulting mixture is stirred well for a minimum of 15 minutes, followed by mechanical agitation at a high shear rate for approximately 5 min, using a probe sonicator at 40% amplitude or a dispersion-tip agitator stirring at high rpm. To the same vessel is added and mixed for 30 minutes using a standard paddle mixer at 700-800 rpm, the following ingredients: pullulan, sucralose, maltodextrin, sorbitol, and dimethyl sulfone. Optionally, the resulting dispersion is degassed while stirring briefly (at least 10 min) under reduced pressure at 15 inHg. Using a mechanical casting apparatus equipped with doctor blade assembly and convection heating chamber, the stable dispersion is cast onto a PET substrate at the rate of 0.55 m/min, followed by a heating in the chamber set at 70-80° C. and 45% RH to remove sufficient water in the composition, thereby producing a uniform thin film having an approximate thickness in the range of 75 to 100 microns. The film was subsequently cut into 0.5 inch by 1.0 inch strips.

Using the ingredients of Table 1 formulated within the ranges shown in the Table 1, the manufacturing an oral dispersible film using this general method involves the formation of a stable wet dispersion, which after casting onto a substrate, produces a thin, semi-opaque film strip with a uniform distribution of active ingredient potency across the film surface. The film will have a low relative standard deviation (RSD) of the concentration of active ingredient, resulting in more accurate dosing and thereby resulting in more consistent therapeutic efficacy, as compared to film strips of the prior art, which do not have methods that use a stable dispersion.

TABLE 1
EXAMPLE 1

| | Amount |
|---|---|
| Ingredient | (% w/w Range) |
| Pullulan | 45-65 |
| Glycerol | 2-12 |
| Tween 80 | 1-5 |
| Span 80 | 1-5 |
| CBD | 10-25 |
| MCT Oil | 1-10 |
| Peppermint Oil | 1-5 |
| Sucralose | 0.01-0.1 |
| Maltodextrin | 1-8 |
| Sorbitol | 0.5-10 |
| Dimethyl Sulfone | 0.05-2 |
| Total | 100 |

### Example 1

### Composition and Method of Making Cannabinoid Oral Dispersible Thin Film

To a 600 mL beaker equipped with magnetic stir bar were added 15 g CBD (99% purity, powder form), 2.5 g MCT oil, and 2.5 g peppermint oil. The mixture was heated at 70° C. while stirring until the mixture produced a homogenous transparent, amber liquid (approximately 30 minutes). Into the same 600 mL beaker was charged 3.5 g glycerol, 4 g Tween 80, 4 g Span 80 and the mixture was stirred at 70° C. until completely dissolved. To this vessel was added 135 g deionized H₂O, the amount of which was sufficient to achieve solids content of approximately 42.5% w/w. The resultant mixture was then sonicated for 5 minutes at 40% amplitude using a probe sonicator. Following sonication, the remaining ingredients were added to the emulsion: 56 g pullulan, 0.07 g sucralose, 5.93 g maltodextrin, 6 g sorbitol, and 0.5 g dimethyl sulfone. The mixture was agitated vigorously using a paddle mixer at 800 rpm for at least 30 minutes, to produce a stable white dispersion. Optionally the stable dispersion was degassed by stirring under reduced pressure at 15 inHg for at least 15 minutes. Using a mechanical casting apparatus equipped with doctor blade assembly and convection heating chamber, the resulting degassed stable dispersion was cast onto a 0.05 mm PET substrate which was heated in a convection style oven at 70° C. (100° C. air temperature) operating at 45% relative humidity, in order to remove H₂O and dry the composition, which deposited a thin film having 85 μm thickness.

### Examples 2-4

### Cannabinoid Oral Dispersible Film With Flavouring Agent

Examples 2-4 are compositions of cannabinoid oral dispersible thin films prepared using the same general method as in Example 1, with the addition of a flavouring agent. The flavouring agent was added in combination with the pullulan, sucralose, maltodextrin, sorbitol, and dimethyl sulfone.

TABLE 2
Compositions of oral dispersible thin films made with cannabinoid
and flavouring agent.

| EXAMPLE 2 | | EXAMPLE 3 | | EXAMPLE 4 | |
|---|---|---|---|---|---|
| Com- | (% | Com- | (% | Com- | (% |
| ponent | w/w) | ponent | w/w) | ponent | w/w) |
| Pullulan | 50 | Pullulan | 50 | Pullulan | 50 |
| Glycerol | 3.5 | Glycerol | 3.5 | Glycerol | 3.5 |
| Tween 80 | 4 | Tween 80 | 4 | Tween 80 | 4 |
| Span 80 | 4 | Span 80 | 4 | Span 80 | 4 |
| CBD | 15 | CBD | 15 | CBD | 15 |
| MCT Oil | 5 | MCT Oil | 5 | MCT Oil | 2.5 |
| Sucralose | 0.072 | Sucralose | 0.072 | Peppermint | 2.5 |
| | | | | Oil | |
| Malto- | 5.928 | Malto- | 5.928 | Sucralose | 0.072 |
| dextrin | | dextrin | | | |
| Sorbitol | 6 | Sorbitol | 6 | Malto- | 5.928 |
| | | | | dextrin | |
| Dimethyl | 0.5 | Dimethyl | 0.5 | Sorbitol | 6 |
| Sulfone | | Sulfone | | | |
| Colourant | 0.5 | Colourant | 1 | Dimethyl | 0.5 |
| | | | | Sulfone | |
| Flavouring | 2.5 | Flavouring | 5 | Colourant | 0.5 |
| Limonene | 3 | | | Flavouring | 5 |
| | | | | Menthol | 0.5 |
| Total | 100 | Total | 100 | Total | 100 |

### Examples 5-6

### Cannabinoid Oral Dispersible Film With Second Active Ingredient

Examples 5 and 6 are prepared according to the general method described in Example 1, except with the addition of a second active ingredient. If the second active ingredient is oleophilic, it can be added simultaneously with the cannabinoid active ingredient, or shortly after the addition of the first active ingredient. If the second active ingredient is hydrophilic, it can be added to the water phase, prior to dispersing with the oil-based mixture. In Example 5, the second active ingredient is melatonin, preferably in the commercially available, powdered form having over 95% purity. In Example 6, the second active ingredient is methylcobalamin, a water-soluble form of vitamin B12; a commercially available, oil-solubilized form methylcobalamin was used.

TABLE 3
Compositions of oral dispersible thin films prepared using the general
method of Example 1, with the addition of a second active ingredient.

| EXAMPLE 5 | | EXAMPLE 6 | |
|---|---|---|---|
| Component | (% w/w) | Component | (% w/w) |
| Pullulan | 50 | Pullulan | 54.8 |
| Glycerol | 3.5 | Glycerol | 3.5 |
| Tween 80 | 4 | Tween 80 | 4 |
| Span 80 | 4 | Span 80 | 4 |
| CBD | 15 | CBD | 15 |
| Melatonin | 6 | Methylcobalamin | 1.2 |
| MCT Oil | 2.5 | MCT Oil | 2.5 |
| Peppermint Oil | 2.5 | Peppermint Oil | 2.5 |
| Sucralose | 0.072 | Sucralose | 0.072 |
| Maltodextrin | 5.928 | Maltodextrin | 5.928 |
| Sorbitol | 6 | Sorbitol | 6 |
| Dimethyl Sulfone | 0.5 | Dimethyl Sulfone | 0.5 |
| Total | 100 | Total | 100 |

### Example 7

### Cannabinoid Oral Dispersible Film with Second Active Ingredient for Animal Health Use

This example was performed using the same general method of Example 1, with the addition of a second active ingredient that is intended for animal health use. If the second active ingredient is oleophilic, it can be added simultaneously with the cannabinoid active ingredient, or shortly after the addition of the first active ingredient. If the second active ingredient is hydrophilic, it can be added to the water phase, prior to dispersing with the oil-based mixture. In Example 7, the second active ingredient is asthaxanthin, a beta carotenoid natural product with many health benefits in dermatology, and an antioxidant with potential anti-inflammatory properties, is obtained commercially as a 35 wt % solution in MCT oil and combined with the CBD in the oil phase mixture of Example 1.

A mucoadhesive strip such as that produced in this example helps deliver the medicinal active agent and prevents the animal from ejecting the medicine (via spitting) which is a common occurence with pills and medicated edibles, etc.

TABLE 4
Compositions of oral dispersible film strips for animal health use,
comprising cannabinoid and a second active ingredient.
EXAMPLE 7
(Animal Health Formula)

| Component | (% w/w) |
|---|---|
| Pullulan | 55 |
| Glycerol | 3.5 |
| Tween 80 | 4 |
| Span 80 | 4 |
| CBD | 15 |
| MCT Oil | 2.5 |
| Peppermint Oil | 2.5 |
| Sucralose | 0.072 |
| Maltodextrin | 5.928 |
| Sorbitol | 6 |
| Dimethyl Sulfone | 0.5 |
| Asthaxanthin | 1.0 |
| (as 35% wt solution | |
| in MCT oil) | |
| Total | 100 |

### Example 8

### Buccal Administration in Mice

Film strips prepared using the method of Example 1 are administered to mice buccal mucosal delivery and compared to oral administration directly in the stomach by gavage. A cannabinoid film strip was manufactured utilizing the method of Example 1 and cut to an appropriate size for delivery of 20 mg/kg of cannabinoid. Mice are weighed the morning of the experiment in order to calculate precise dose of the cannabinoid oral thin film strip. Mice are gently restrained and the strip is placed inside the mouth of the mice, against the buccal tissue, held in place with forceps until softened. The serum levels of cannabinoid are measured prior to administration, and at 1 minute, 5 minutes, 10 minutes, 30 minutes, and 60 minutes after administration. The serum levels of cannabinoid are compared to those of two control groups of mice, where an identical amount of cannabinoid was delivered via (a) oral administration by gavage directly into the stomach; and (b) oral dissolvable thin strip manufactured using a prior art methodology. It is found that the cannabinoid delivered by the oral dispersible thin film of the present invention entered the blood stream is more rapidly delivered than control group (a) by at least 1.3 to 1.8 times. Cannabinoids are detected in the serum of mice as quickly as 1 minute post-administration, with a peak appearing within 5-10 minutes post-administration—significantly faster than the peak (approximately 30 minutes post-administration) of control group (a) mice.

Serum cannabinoid concentrations are also significantly higher as compared to control group (a), with an approximately 1.5 to 3 fold increase in serum cannabinoid concentration utilizing the film strips of the present invention and manufacturing methodology, compared to oral gavage administration. In addition, and perhaps more significantly, the variation in level of serum cannabinoid concentration in different mice (i.e. deviation across samples) is significantly lower in the film strips made using the present methodology, as compared to prior art film strip technology, indicating that the stable dispersion allows for more even dosing across the film strip manufacture.

### Example 9

### Analytical Qualification of the Oral Dispersible Thin Films Produced in Examples 1-7 and Comparative Examples

The products were analytically qualified by analyzing the potency with high performance liquid chromatography (HPLC), dissolution time, and physical dimensions.

HPLC analysis was performed using a Waters® 600E high-performance liquid chromatograph pump, 727 plus autosampler and 2996 photodiode array detector. The cannabinoids (analyte) were extracted from the oral thin film using 10 mL of HPLC-grade MeOH for 3 hours, diluted with 80% MeOH in H₂O (v/v), and filtered using a 0.22 μm nylon syringe filter prior to loading on the HPLC column. The cannabinoid analytes were separated at 25° C. using a Phenomenex® C18 2.6 μm 100 Å, 4.6 mm×150 mm column and a gradient method with the following solvents: Solvent A) 0.1% formic acid in H₂O; Solvent B) 0.1% formic acid in acetonitrile. The 30:70% Solvent A:B gradient changed to 5:95% over 9.00 min with a flow rate of 1.0 mL/min. The active agents were quantified at 228 nm using Empower® 2 software to process the data.

The CBD amount was quantified at a retention time of 6.1 min, and Δ9-THC was quantified at 11.4 min using the cannabinoid HPLC method. The quantification of the cannabinoids was based on the calibration performed using Cerilliant® cannabinoid USP secondary standards.

The secondary active agent in Example 5 (melatonin) was quantified using an isocratic method with 60:40 ratio of [0.1% formic acid in H₂O]:[0.1% formic acid in MeOH] at 0.5 mL/min and 25° C. The retention time of the melatonin peak was 8.9 min and the total run time was 11.0 min. A USP secondary standard obtained from Sigma-Aldrich® was used to calibrate the method for melatonin quantification.

The potency of CBD or active ingredient in the oral thin film strips was determined based on the average percentage of active agent in the strip by weight (w/w), and the uniformity of the dosage unit was measured using the percent relative standard deviation (% RSD) (see Table 5). The percentage of the active agent in the strip was calculated by dividing the active agent quantity determined using HPLC by the mass of the oral thin film. The % RSD corresponds to the standard deviation of the potency of the analyzed oral thin films divided by the average potency. The analyzed samples were selected randomly.

TABLE 5
Analytical qualification of the product using potency
determined by HPLC analysis.

| | | % Active Agent | % RSD of Active |
|---|---|---|---|
| Strip Type | Active Agent | in Strip (w/w) | Agent in Strip |
| Example 1 | CBD | 13.4% | 0.2% |
| Example 2 | Δ9-THC | 12.2% | 0.8% |
| Example 5 | CBD | 13.2% | 0.8% |
| | Melatonin | 5.5% | 0.4% |
| Comparative | CBD | 15.0% | |
| Example 1 | | | 0.3% |
| Comparative | CBD | 13.0% | |
| Example 2 | | | 0.2% |

### Example 10

### Dissolution Time For Examples

Oral thin film dissolution in vitro was evaluated in a solution of phosphate buffered saline (PBS; pH 6.8) at 37° C. to mimic the oral cavity. The oral thin film was submerged in the PBS using forceps and quickly released. The solution was stirred using a stir bar at 400 rpm to stimulate movement in the solution. The stopwatch was started upon film submersion and stopped upon complete dissolution, such that no particles remained suspended in the solution. The procedure was completed for 10 replicates of each oral thin film example. The reported dissolution times are the averages of the replicate measurements and the error corresponds to the standard deviation (Table 6).

The results for dissolution time in Table 6 indicate that the examples 1, 2 and 5, which are oral dispersible thin films made according to the method of the present invention had on average shorter in vitro dissolution times, ranging from 25 to 45 seconds, than the Comparative Example 1 which is a formula and method of making oral thin films according to U.S. Pat. No. 10,265,362, which gave an average dissolution time in our hands of 62 seconds. Comparative Example 2 is an oral dispersible thin film comprising the formula of the present invention (that is, it is similar to the formula of Example 1) but prepared using the method of making reported in prior art U.S. Pat. No. 10,265,362. The dissolution time for Comparative Example 2 is within the same range of dissolution times for the oral thin film compositions of this invention (as found in Examples 1, 2, 5) which indicates that both composition AND method of making oral thin film strip of the present invention are important and necessary factors to achieve rapid delivery and high bioavailability of active agents via oral dispersible thin film.

TABLE 6
Dissolution times (in vitro) for Examples 1, 2, 5
and Comparative Examples 1 and 2.

| Oral Thin Film | Average |
|---|---|
| Preparation | Disintegration Time (seconds) |
| Example 1 | 36 ± 14 |
| Example 2 | 26 ± 5 |
| Example 5 | 44 ± 12 |
| Comparative Example 1 | 62 ± 24 |
| Comparative Example 2 | 36.0 ± 7 |

### Example 11

### Particle Size Characterization of Oral Dispersible Thin Films

The oral dispersible thin films of the Examples 1,2 and 5, and Comparative Examples 1 and 2, were characterized by optical microscopy imaging for average particle size within the thin film single layer composition. The images were captured on an Olympus CKX41 optical microscope with a Olympus Q-Color 3 Imaging System, and rendered into images using QCapture Pro imaging software. The method for sample preparation requires selecting three (3) strips from each batch sample of oral thin film strips, which were each imaged once. A minimum of 15 particles were measured per strip. For particles having an ellipsoidal shape, the smaller diameter was the measurement taken for analysis. The results for average particle size, standard deviation and PDI values are summarized in Table 7 below. The results indicate that the average particle sizes and PDI are lower in the Examples 1,2,5 oral thin films made by the present invention, as compared with the oral thin films made in the Comparative Examples 1 and 2. Moreover, the results for Comparative Example 1, which are oral dispersible films prepared with the formula composition and method of making according to U.S. Pat. No. 10,265,362, shows the highest average particle size and highest PDI value amongst all the samples analyzed in the table.

The data strongly indicates that both the if the formula composition AND method of making oral dispersible thin films by the present invention are conducted, the highest quality thin films are prepared, which in turn will have a high potential for rapid oral mucosal delivery of the active ingredient, as well as higher bioavailability in blood serum, as evidenced in the results of Example 8 describing the bioanalytical testing in mice.

TABLE 7
Average particle sizes of oral dispersible thin films in Examples
1, 2, 5 and Comparative Examples 1, 2, measured by optical
microscopy imaging. (PDI is polydispersity index, which is a
measure of the width of the distribution of particle sizes.
The closer the number is to 1.00, the narrower the particle size
distribution, with PDI value of 1.00 representing
monodisperse particles (all have exactly the same particle size).

| | Average | | | |
|---|---|---|---|---|
| Oral Dispersible Film | Size | | | |
| Preparation | (μm) | St. Dev. | PDI | St. Dev. |
| COMPARATIVE EXAMPLE 1 | 5.629 | 0.793 | 2.437 | 0.635 |
| COMPARATIVE EXAMPLE 2 | 4.591 | 0.960 | 1.420 | 0.269 |
| EXAMPLE 1 | 4.089 | 1.086 | 1.505 | 0.065 |
| EXAMPLE 5 | 4.027 | 0.384 | 1.337 | 0.097 |
| EXAMPLE 2 | 4.681 | 0.048 | 1.257 | 0.030 |

1. An oral dispersible film composition comprising at least one active ingredient and a film forming agent, wherein the active ingredient is dispersed within the film and has an average particle size of 1-20 microns, preferably 5-15 microns, wherein the composition consists of a single layer.

2. An oral dispersible film composition comprising at least one active ingredient and a film forming agent, having a percentage relative standard deviation of potency concentration of the at least one active ingredient in a batch of said oral dispersible film composition of less than 2.5%, preferably less than 1%, more preferably less than 0.5%.

3. An oral dispersible film composition comprising at least one active ingredient and a film forming agent, having a dissolution time in phosphate buffered saline solution at pH 6.8 and 37 degrees C. of less than about 1 minute, preferably less than about 30 seconds, more preferably less than about 20 seconds.

4. The oral dispersible film composition of any preceding claim where the distribution of particle sizes have a polydispersity value of less than 1.5.

5. The oral dispersible film composition of any preceding claim wherein the composition contains less than 1 wt %, more preferably less than 0.5 wt %, most preferably is free of a gum resin.

6. The oral dispersible film composition of any preceding claim wherein the at least one active ingredient is selected from the group of cannabinoid compounds and terpenoid compounds comprised of cannabidiol, delta-9-tetrahydrocannabinol, delta-8-tetrahydrocannabinol, cannabigerol, cannabidiol acid, tetrahydrocannabinol acid, olivetol and olivetol acid or esters.

7. The oral dispersible film composition of any preceding claim wherein the at least one active cannabinoid compound is derived biosynthetically from Cannabis plant species, from hemp, from plant-based or animal cell microorganisms, or is obtained by chemical synthesis from non-natural starting compounds.

8. The oral dispersible film composition of any preceding claim comprising a second active ingredient.

9. The oral dispersible film composition of claim 8 wherein the second active ingredient is a nutraceutical or natural health product selected from the group consisting of vitamin D3 (cholecalciferol), docosahexenoic acid (DHA), caffeine, nicotine, ubiquinone (coenzyme Q10), curcumin, natural antioxidants, glucosamine, melatonin, vitamin B12, a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

10. The oral dispersible film composition of any preceding claim wherein the film forming agent is selected from the group consisting of pullulan, substituted and modified cellulosic polymers such as HPMC and CMC, alginate salts, starches, pectins, dextrins, gelatins, glycogen, poly(vinylalcohol) and its derivatives including polyvinylacetate, polyethyleneoxide, polyethyleneglycol, polyvinylpyrrolidone (povidone), and the oral film composition preferably contains less than 1 wt %, more preferably less than 0.5 wt %, most preferably 0 wt % of a gum resin.

11. An oral dispersible film composition comprising pullulan, a cannabinoid, glycerol, Tween 80, Span 80, MCT oil, peppermint oil, sucralose, maltodextrin, sorbitol, dimethyl sulfone, and optionally one or more of colourant, flavouring, limonene and menthol.

12. The oral dispersible film composition of any one of the preceding claims wherein the active cannabinoid ingredient is CBD (cannabidiol) or delta-9-THC (delta-9-tetrahydrocannabinol), or blends of CBD and THC, preferably present in a relative weight ratio of between 20:1 to 1:5 (CBD:THC).

13. An oral dispersible film composition, consisting essentially of: a pharmaceutical-grade or food-grade plasticizer (preferably glycerol), a cannabinoid (preferably CBD or delta-9-THC or a blend of CBD and THC in a relative weight ratio of between 20:1 to 1:5), a carrier fluid, a gum-free film forming agent (preferably pullulan), and optionally one or more of a colourant, a flavouring agent, a sweetener, one or more surfactants (preferably non-ionic surfactant), and a permeation enhancer.

14. The oral dispersible film composition of claim 13 consisting essentially of 3-5 wt % plasticizer, 12-18 wt % cannabinoid, 2-8 wt % carrier oil, 40-65 wt % gum-free film forming agent, 4-12 wt % surfactant, 0.1-1 wt % permeation enhancer, and optionally colourant, flavouring, sweetener, limonene and menthol.

15. The oral dispersible film composition of claim 13 or 14 further consisting essentially of one or more of vitamin D3 (cholecalciferol), docosahexenoic acid (DHA), caffeine, nicotine, ubiquinone (coenzyme Q10), curcumin, natural antioxidants, glucosamine, melatonin, vitamin B12, a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

16. The oral dispersible film composition of any one of the preceding claims wherein the cannabinoid is delta-9-THC.

17. The oral dispersible film composition of any one of the preceding claims wherein the cannabinoid is CBD.

18. The oral dispersible film composition of any one of the preceding claims wherein the composition consists of a single layer.

19. The oral dispersible film composition of any one of the preceding claims wherein the at least one active ingredient is CBD and the second active ingredient is vitamin B12, a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

20. The oral dispersible film composition of any one of the preceding claims wherein the at least one active ingredient is CBD and the second active ingredient is melatonin.

21. A method of treating active inflammatory bowel disease in a patient in need thereof comprising administering a therapeutically effective amount of an oral dispersible film composition of claim 17 or claim 19.

22. A method of treating a sleep disorder or a sleep disturbance such as sleep apnea and insomnia, in a patient in need thereof comprising administering a therapeutically effective amount of an oral dispersible film composition of claim 20.

23. A use of an oral dispersible film composition of claims 12 and 16 for the treatment of dysfunctional anxiety and pain in patients suffering from post-traumatic stress disorder and similar mental health conditions.

24. Method of manufacturing an oral dispersible film comprising a first active medicinal ingredient, said method comprising:
combining the first active ingredient with a carrier fluid, and blending to form a mixture;
adding at least one surfactant to the mixture;
agitating the mixture at a high shear rate to create a stable dispersion;
adding a polymer film-forming agent, a plasticizer, a permeation enhancer, a diluent, optionally at least one flavouring agent, and optionally at least one colorant
casting the stable dispersion on a flexible substrate having thickness in the range of 0.02 mm to 0.08 mm;
drying the dispersion on the flexible substrate at a local temperature ranging from 70° C. to no more than 100° C. and relative humidity ranging from 40% to about 55%, to form the oral dispersible thin film.

25. The method of claim 24 wherein the agitating at a high shear rate results in the mixture having an average particle size of the one or more active ingredient being in the range of 1 to 20 microns, and a polydispersity value of less than 1.5.

26. Method of any one of the preceding claims wherein the first active ingredient is a cannabinoid.

27. The method of claim 26 wherein the first active ingredient is CBD.

28. The method of claim 26 wherein the first active ingredient is THCA.

29. The method of claim 26 wherein the first active ingredient is THC.

30. The method of any one of the preceding claims, wherein the oral dispersible film comprises a second active ingredient, said method comprising combining the second active pharmaceutical ingredient with the carrier before, simultaneously with, or after, the combining of the first active pharmaceutical ingredient with the carrier.

31. The method of claim 30, wherein the second active ingredient is selected from the group consisting of vitamin D3 (cholecalciferol), docosahexenoic acid (DHA), caffeine, nicotine, ubiquinone (coenzyme Q10), curcumin, natural antioxidants, glucosamine, melatonin, vitamin B12, a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

32. The method of claim 31 wherein the second active ingredient is melatonin.

33. The method of claim 31 wherein the second active ingredient is vitamin B12 or methylcobalamin.

34. The method of claim 30 wherein the first active ingredient is CBD and the second active ingredient is melatonin.

35. The method of claim 30 wherein the first active ingredient is CBD and the second active ingredient is vitamin B12, a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

36. The method of any one of the preceding claims wherein the polymer film-forming agent comprises less than 1 wt %, more preferably less than 0.5 wt % of gum resin, and even more preferably is free of gum resin.

37. An oral dispersible film manufactured utilizing the method of any one of the preceding claims.

38. An oral dispersible film manufactured utilizing the method of any one of the preceding claims, wherein the particle dispersion within the film has an average particle size of 1-20 microns, preferably 5-15 microns.

39. An oral dispersible film composition manufactured utilizing the method of any one of the preceding claims, having a percentage relative standard deviation of potency concentration of the at least one active ingredient in a batch of said oral dispersible film composition of less than 2.5%, preferably less than 1%, more preferably less than 0.5%.

40. An oral dispersible film composition manufactured utilizing the method of any one of the preceding claims, having a dissolution time in phosphate buffered saline solution at pH 6.8 and 37 degrees C. of less than about 1 minute, preferably less than about 30 seconds, more preferably less than about 20 seconds.

41. An oral dispersible film composition manufactured utilizing the method of any one of the preceding claims, wherein the sizes of particles dispersed in the film have a polydispersity value of less than 1.5.

42. Use of an oral dispersible film composition manufactured utilizing the method of any one of the preceding claims in the treatment of active inflammatory bowel disease, wherein the active ingredient is CBD and the optional second active ingredient is vitamin B12, a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

43. Use of an oral dispersible film composition manufactured utilizing the method of any one of the preceding claims in the treatment of a sleep disorder or a sleep disturbance, wherein the active ingredient is CBD and the optional second active ingredient is melatonin.

44. Use of an oral dispersible film composition of any one of the preceding claims in the treatment of active inflammatory bowel disease, wherein the active ingredient is CBD and the second active ingredient is vitamin B12, a biologically active metabolite thereof such as methylcobalamin, iron, any essential vitamin and essential mineral and derivative forms thereof such as a therapeutically acceptable inorganic salt, a therapeutically acceptable organic salt, a polymer-bound complex, and a protein-bound complex.

45. Use of an oral dispersible film composition of any one of the preceding claims in the treatment of a sleep disorder or a sleep disturbance, wherein the active ingredient is CBD and the second active ingredient is melatonin.

46. An oral dispersible film composition of any one of the preceding claims, or an oral dispersible film composition manufactured utilizing the method of any one of the preceding claims, having improved bioavailability by a factor of 1.5 or more compared to oral administration directly into the GI tract.
